(19) United States
(12) Patent Application Publication
(10) Pub. No.: US 2012/0150107 A1
(43) Pub. Date: Jun. 14, 2012

(54) BALLOON CATHETER SHAFTS AND METHODS OF MANUFACTURING

(75) Inventors: DESMOND CHEUNG, SAN JOSE, CA (US); RAJ SUBRAMANIAM, FREMONT, CA (US); ZAYA TUN, LIVERMORE, CA (US)

(73) Assignee: BOSTON SCIENTIFIC SCIMED, INC., MAPLE GROVE, MN (US)

(21) Appl. No.: 13/312,755

(22) Filed: Dec. 6, 2011

Related U.S. Application Data
(60) Provisional application No. 61/423,009, filed on Dec. 14, 2010.

(51) Int. Cl. A61M 29/00; B23P 11/02

(52) U.S. Cl. 604/96.01; 29/447

(57) ABSTRACT

Balloon catheters and methods for manufacturing catheter shafts are disclosed. In one example, a balloon catheter may include an outer tubular member disposed about an inner tubular member. In some cases, a fluid (e.g. cryogenic fluid) can be provided between the inner tubular member and the outer tubular member and may cool the inner tubular member. To help provide a relatively uniform temperature distribution around a circumference of the inner tubular member, two or more spacers, protrusions, or gap-maintaining members can be positioned between the outer tubular member and the inner tubular member to maintain a gap therebetween. In some cases, the spacers, protrusions, or gap-maintaining members may be attached to an inner surface of the outer tubular member or, in other cases, to an outer surface of the inner tubular member. In other embodiments, a step-down in an outer diameter of the outer tubular member is disclosed.

### CROSS-REFERENCE TO RELATED APPLICATIONS

This application claims the benefit of U.S. Provisional Application Ser. No. 61/423,009, filed Dec. 14, 2010, the entire disclosure of which is incorporated herein by reference.

### FIELD

The present disclosure relates generally to medical devices and, more particularly, to balloon catheter shafts.

### BACKGROUND

A variety of minimally invasive electrophysiological procedures employing catheters and other apparatuses have been developed to treat conditions within the body by ablating soft tissue. With respect to the heart, minimally invasive electrophysiological procedures have been developed to treat atrial fibrillation, atrial flutter and ventricular tachycardia by forming therapeutic lesions in heart tissue. The formation of lesions by the coagulation of soft tissue (also referred to as “ablation”) during minimally invasive surgical procedures can provide the same therapeutic benefits provided by certain invasive, open heart surgical procedures.

For some of these procedures, a catheter, such as an ablation catheter, is typically advanced into the heart via the patient's vessels to deliver the desired therapy. Some ablation catheters can employ electrodes for delivering radio frequency (RF) energy to the soft tissue to form the desired lesions. Other ablation catheters can employ a balloon for delivering cryotherapy or extracting heat, through the surface of the balloon, from the soft tissue to form the lesions. In these cryotherapy procedures, a cooling fluid (e.g. cryogenic fluid) flowing through the catheter can, in some instances, cause freezing of a fluid (e.g. blood) in one or more lumens of the catheter, such as the guidewire lumen. Ice build-up from the freezing fluid can, in some situations, rupture the lumen. Therefore, there is a need for new and improved balloon catheter shafts.

### BRIEF SUMMARY

The present disclosure relates generally to catheters and, more particularly, to balloon catheter shafts. In one illustrative embodiment, a catheter may include an outer tubular member, an inner tubular member, and two or more spacers or protruding members therebetween. The outer tubular member may include a proximal region, a distal region, and a lumen extending therethrough. The inner tubular member may include a proximal region, a distal region, and a lumen extending therethrough. The inner tubular member may be at least partially disposed in the lumen of the outer tubular member. The two or more spacers or protruding members may be configured to maintain a gap between the inner tubular member and the outer tubular member to, in some cases, provide a generally uniform temperature distribution for the inner tubular member. In some cases, a balloon assembly can be coupled to the distal region the outer tubular member and the distal region of the inner tubular member.

In some embodiments, three or more spacers or protruding members can be provided. The spacers or protruding members may be positioned on an inner surface of the outer tubular member and/or on an outer surface of the inner tubular member. In some cases, at least one of the two or more spacers or protruding members may include conduits disposed therethrough.

In some cases, the outer tubular member may include a step-down in outer diameter in the distal region while maintaining a substantially constant inner diameter.

In another illustrative embodiment, a method of manufacturing a catheter body is disclosed. The method may include assembling a multi-lumen outer tubular member including an inner liner, a reinforcement layer disposed over the inner liner, and an outer layer disposed over the reinforcement layer. The multi-lumen outer tubular member may include two or more conduits disposed between the inner liner and the reinforcement layer and the two or more conduits may have a higher melting temperature than the inner liner and the outer layer. The two or more conduits can also form two or more radial protrusions on an inner surface of the multi-lumen outer tubular member. The method may also include reflowing the inner liner and the outer layer and disposing an inner tubular member within the multi-lumen outer tubular member to define a cooling lumen therebetween. In this example, the two or more radial protrusions on the inner surface of the multi-lumen inner tubular member can be configured to maintain a gap between the inner tubular member and the multi-lumen outer tubular member.

In another illustrative embodiment, a method of performing a cryoablation procedure with a catheter is disclosed. The method may include providing a catheter shaft including an inner tubular member and an outer tubular member, where a lumen is defined between the inner tubular member and the outer tubular member. The method can also include providing a fluid that has a relatively cool temperature in the lumen and maintaining a substantially uniform temperature distribution in the inner tubular member. In some cases, the substantially uniform temperature distribution may be maintained in the inner tubular member by providing three or more protruding members on an inner surface of the outer tubular member and/or an outer surface of the inner tubular member.

The preceding summary is provided to facilitate an understanding of some of the innovative features unique to the present disclosure and is not intended to be a full description. A full appreciation of the disclosure can be gained by taking the entire specification, claims, drawings, and abstract as a whole.

### BRIEF DESCRIPTION OF THE DRAWINGS

The disclosure may be more completely understood in consideration of the following detailed description of various illustrative embodiments of the disclosure in connection with the accompanying drawings, in which:

FIG. 1 is a perspective view of an illustrative embodiment of a balloon catheter;

FIG. 2 is an illustrative transverse cross-sectional view of the balloon catheter of FIG. 1 taken along line A-A;

FIG. 3 is another illustrative transverse cross-sectional view of the balloon catheter of FIG. 1 taken along line A-A;

FIG. 4 is a perspective view of the transverse cross-sectional shown in FIG. 3;

FIG. 5 is a side view of the catheter shaft of FIG. 1;

FIG. 6 is a cross-sectional side view of an illustrative balloon assembly that may be employed by the balloon catheter of FIG. 1;

FIG. 7 is a perspective view of an illustrative embodiment of a mandrel that may be used in manufacturing the balloon catheter of FIG. 1;

FIG. 8 is a cross-sectional side view of an illustrative distal region that may be used in the balloon catheter shown in FIG. 1;

FIG. 9 is a cross-sectional side view of another illustrative distal region that may be used in the balloon catheter shown in FIG. 1; and

FIG. 10 is another illustrative transverse cross-sectional view of the balloon catheter of FIG. 1 taken along line A-A.

### DETAILED DESCRIPTION

The following description should be read with reference to the drawings wherein like reference numerals indicate like elements throughout the several views. The detailed description and drawings, which are not necessarily drawn to scale, show several embodiments which are meant to be illustrative and are not intended to limit the scope of the disclosure.

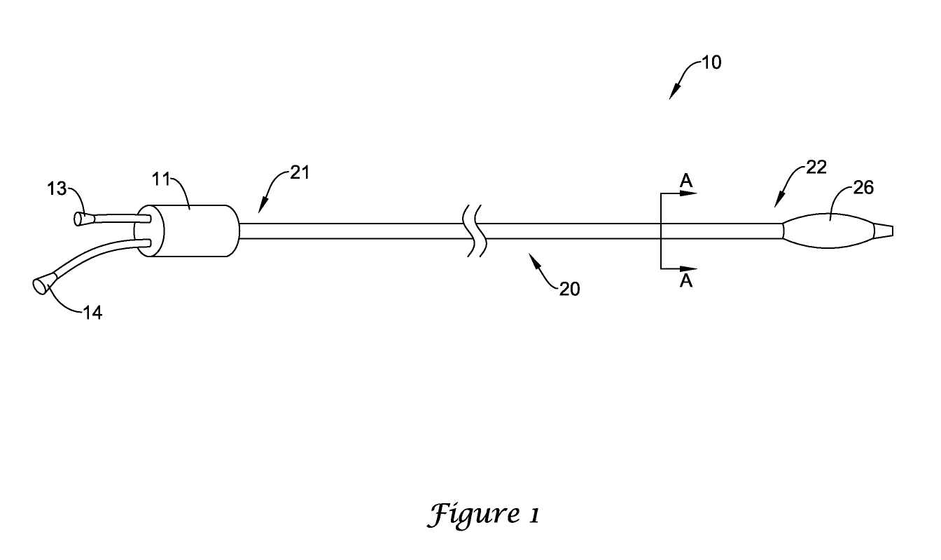

FIG. 1 is an illustrative embodiment of a balloon catheter 10 in accordance with one aspect of the present disclosure. In the illustrative embodiment, the balloon catheter 10 may include a catheter shaft 20 having a proximal region 21 and a distal region 22. As shown in FIG. 1, a balloon assembly 26 is disposed about the distal region 22 of catheter shaft 20. In some embodiments, a fluid, such as a cooling or cryogenic fluid, can be delivered to the proximal region 21 of the catheter shaft 20 and may flow through the catheter shaft 20 and into the balloon assembly 26 to expand the balloon assembly 26 for cryoablating adjacent tissue.

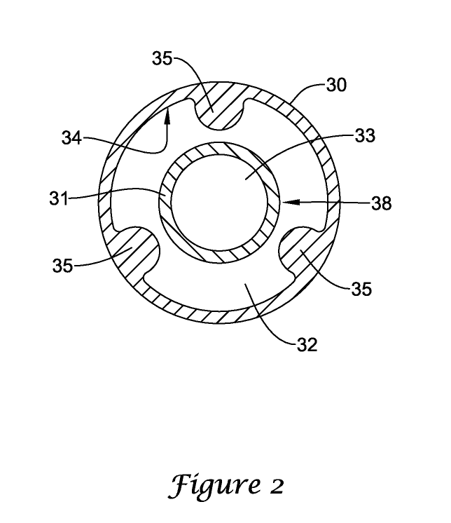

As shown in FIG. 1, the balloon catheter 10 may include a hub 11 coupled to the proximal region 21 of the catheter shaft 20. Hub 11 may be configured to facilitate coupling of the balloon catheter 10 to external equipment. For example, hub 11 may include a port 13 for connecting a cryogenic fluid source to an inflation lumen (shown as 32 in FIG. 2) of the catheter shaft 20. In some embodiments, the balloon catheter 10 may be an over-the-wire cryotherapy balloon catheter and the balloon catheter 10 may be advanced over a guidewire (not shown) to a desired location within a patient. In this embodiment, hub 11 may also include a port 14 connected to a guidewire lumen (shown as 31 in FIG. 2) of the catheter shaft 20 for receiving the guidewire therethrough. In some cases, the guidewire lumen 31 may extend distally of the balloon assembly 26, as shown. It is contemplated that the hub 11 may include additional ports that are fluidly connected to additional lumens, such as, for example, vacuum lumens, sensor lumens (e.g. pressure, temperature, etc.), and/or other lumens or combinations thereof. Further, the foregoing hub 11 is merely illustrative and is not meant to be limiting in any manner. It is contemplated that other suitable hubs or port component configurations may be used, as desired.

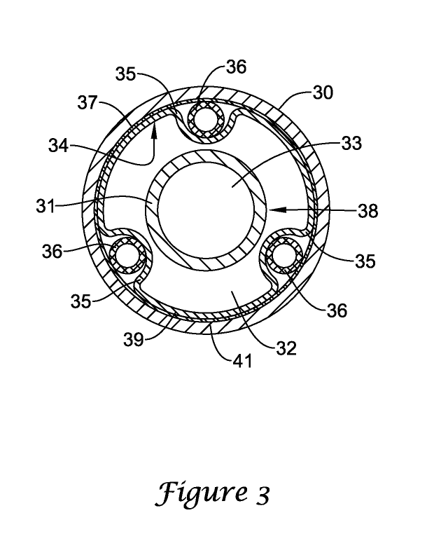

In the illustrative embodiment, the length, diameter, and flexibility of the balloon catheter 10 help enable the balloon catheter 10 to be inserted into a desired portion of the body. In some examples, the balloon catheter 10 may be about 6 French to about 10 French in diameter and the portion of the balloon catheter 10 that is inserted in other patient may be from about 60 to about 160 cm in length. However, these dimensions are merely illustrative and it is contemplated that the balloon catheter 10 may have any desired diameter and/or length. In some embodiments, the catheter shaft 20 may be manufactured to have a variable stiffness along the length of the catheter shaft 20. For example, the proximal region 21 of the catheter shaft 20 may be configured to be stiffer than the distal region 22 of the catheter shaft 20. In some instances, the variable stiffness may be imparted into the catheter shaft 20 by varying the durometer of polymers used to manufacture the catheter shaft or by varying the pitch of a reinforcement layer (e.g. coil or braid), such as reinforcement layer 37 shown in FIG. 3. However, other techniques for varying the stiffness in the catheter shaft 20 may also be used. In some cases, the catheter shaft may include an intermediate region, such as a midshaft region, between the proximal region 21 and the distal region 22 that is configured to provide a gradual transition in stiffness between the proximal region 21 and the distal region 22. For some applications, the variable stiffness catheter shaft 20 may, for example, help provide smoother transitions, better trackability, and/or better pushability.

FIGS. 2 and 3 are illustrative transverse cross-section views of the illustrative catheter shaft 20 taken along line A-A in FIG. 1. As shown in FIG. 2, the catheter shaft 20 includes an outer tubular member 30 and an inner tubular member 31 disposed within the outer tubular member 30. An inflation lumen 32 may be defined between the inner surface of the outer tubular member 30 and the outer surface of inner tubular member 31. The inflation lumen 32 may be configured to be in fluid communication with the inflatable balloon assembly 26 (shown in FIG. 1) and a cooling fluid supply (not shown) in order to supply cooling fluid (e.g. cryogenic fluid) to the balloon assembly 26. However, in other embodiments, it is contemplated that lumen 32 may be an exhaust lumen configured to exhaust fluid from the balloon assembly 26, if desired. In this embodiment, the balloon catheter 10 may include a supply lumen (not shown) to deliver fluid (e.g. cryogenic fluid) from external source to an interior chamber of the balloon assembly 26. In some cases, a distal end of the supply lumen may include one or more orifices (not shown) configured to release the cryogenic fluid in the interior chamber of the balloon assembly 26. When so provided, at least some of the cryogenic fluid can undergo a liquid-to-gas phase change when released in the interior chamber that cools the balloon assembly 26 by the Joule-Thomson effect. Gas resulting from the cryogenic fluid being released inside the chamber can be exhausted through inflation lumen, such as lumen 32, which may serve as an exhaust lumen.

In the illustrative embodiment, the inner tubular member 31 may define an inner lumen 33 extending therethrough, which is configured to slidably receive a guiding element (e.g. guidewire or the like) to facilitate guiding of the balloon catheter 10 to a target location within patient. The inner lumen 33 (e.g. guidewire lumen) may be formed from any flexible material (e.g., a thermoplastic, or the like) that maintains elasticity over a wide range of temperatures, particularly at a temperature of the cooling fluid.

In the embodiment illustrated in FIG. 2, an inner surface 34 of the outer tubular member 30 may include one or more protrusions, bumps, or spacers 35 (hereinafter referred to as protrusions) that extend along the inner surface 34 of the outer tubular member 30 and that protrude or extend radially into the inflation lumen 32. The one or more protrusions 35 may be configured to function as gap-maintaining members or, in other words, to maintain a distance between the inner surface 34 of the outer tubular member 30 and an outer surface 38 of the inner tubular member 31. The one or more protrusions 35 may help to prevent the inner tubular member 31 from contacting the outer tubular member 30. As shown in FIG. 2, there are three protrusions 35. However, it is contemplated that there may be two or more protrusions, three or more protrusions, four or more protrusions, or any other number of protrusions, as desired.

Although not shown in FIG. 2, the one or more protrusions 35 may be configured to extend along a length of the catheter shaft 20, a length of the outer tubular member 30, and/or a length of the inner tubular member 31. For example, the one or more protrusions 35 may be configured to extend along an entire length of the outer tubular member 30. In other examples, the one or more protrusions 35 may be configured to extend along only a portion of the length of the outer tubular member 30, such as, for example, about 10 percent of the length, about 20 percent of the length, about 25 percent of the length, about 50 percent of the length, about 60 percent of the length, about 75 percent of the length, about 85 percent of the length, about 95 percent of the length, or any other percent of the length of the outer tubular member 30, as desired. Further, in some cases, the catheter shaft 20 may have a length that is similar to the length of the outer tubular member 30.

In the illustrative embodiment, the one or more protrusions 35 may help to provide a more uniform temperature distribution along the circumference of inner tubular member 31. For example, if the protrusions 35 are not included in the catheter body 20, the inner tubular member 31 could contact the outer tubular member 30 and, when this occurs, the portion of the inner tubular member 31 contacting the outer tubular member 30 may be exposed to a warmer temperature than the remainder of the inner tubular member 31 due to the cooling fluid (e.g. cryogenic fluid) flowing through inflation lumen 32. In some cases, this can cause a non-uniform temperature distribution throughout the circumference of the inner tubular member 31. In this instance, ice may have a tendency to form in the portion of the inner tubular member 31 having a colder temperature (e.g. portion of the inner tubular member 31 that is not contacting the outer tubular member 30). When the ice builds up, the force of volume expansion due to the ice formation may be more focused at a point or portion of the inner tubular member 31 that is contacting the outer tubular member 30 and may eventually cause the inner tubular member 31 to rupture or crack. Such a rupture or crack may allow cooling fluid (e.g. cryogenic fluid) to leak into the guidewire lumen 33 of the balloon catheter 10. By keeping the inner tubular member 31 generally centered in the outer tubular member 30, or at least spaced from the outer tubular member 30 so that fluid can flow on all sides of the inner tubular member, the inner tubular member 31 may have a generally uniform temperature distribution. In some cases, the generally uniform temperature distribution may more evenly distribute any ice formations around the circumference of the lumen 33. The generally uniform formation of ice may, in some cases, also more evenly distribute expansion forces around the inner wall of the inner tubular member 31 thereby decreasing the likelihood of rupture of the inner tubular member 31.

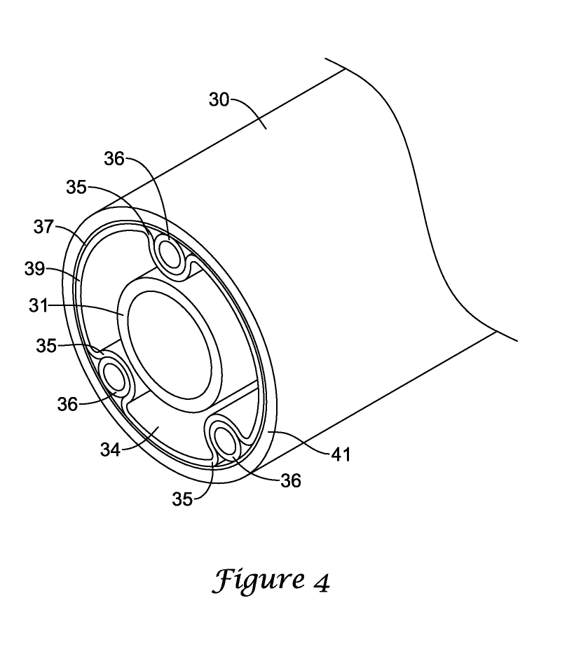

FIGS. 3 and 4 show other illustrative transverse cross-section of a catheter body 20 taken along line A-A of FIG. 1 that may be employed by the catheter shaft of FIG. 1. In particular, FIG. 4 is a perspective view of the catheter body 20 shown in FIG. 3. Similar to FIG. 2, the embodiment of FIGS. 3 and 4 includes the outer tubular member 30 and the inner tubular member 31 disposed within the outer tubular member 30. The inflation lumen 32 can be defined between the outer tubular member 30 and the inner tubular member 31. Similar to FIG. 2, protrusions 35 can be configured to maintain a gap, or a portion of the inflation lumen 32, between the outer tubular member 30 and the inner tubular member 31.

In the illustrative embodiment shown in FIGS. 3 and 4, one or more conduits 36 may be provided in at least one of the one or more protrusion 35. As shown, each protrusion 35 may include a conduit 36, but this is not required. It is contemplated that only some of protrusions 35 may include conduits, if desired. The one or more conduits 36 may be configured to transport fluid, sense parameters (e.g. pressure, temperature, vacuum, etc.) and/or route electrical wires and/or sensors through the catheter shaft 20. For example, the one or more conduits 36 can include a pressure monitoring lumen for controlling and/or monitoring the pressure with the balloon assembly 26, a vacuum lumen, a supply lumen, and/or any other suitable lumen, as desired. While three protrusions 35 and conduits 36 are shown in FIG. 3, it is contemplated other numbers of protrusions 35 and conduits 36 may be used and, also, that conduits 36 may or may not run through each protrusion 35, as desired.

In the illustrative embodiment, the catheter shaft 20 may include an outer layer 41, a reinforcement layer 37, the one or more conduits 36, and an inner liner 39. The outer layer 41, reinforcement layer 37 and/or the inner liner 39 may be reflowed to form a multi-lumen catheter shaft. In some cases, the outer layer 41 and the inner liner 39 may include the same or different materials. However, in any event, the outer layer 41 and the inner liner 39 may be formed of suitable materials typically employed in catheter shafts. Example materials may include, for example, a polymer including but not limited to polyolefin copolymer, polyester, polyethylene teraphthalate, polyethylene, polyether-block-amide, polyamide (e.g. nylon), polytetrafluoroethylene (PTFE), polyimide, latex, a urethane-family material, neoprene, etc. An example polyether-block-amide is available under the trade name PEBAX®. However, the foregoing materials are merely illustrative and it is contemplated that any suitable materials may be used, as desired.

In the illustrative embodiment, the reinforcement layer 37 may help to support the catheter shaft 20 and reduce kinking In some cases, the reinforcement layer 37 may include a coil or a braid. However, other suitable components may be used, as desired. Example materials that may be used in the reinforcement layer can include metals, metal alloys, polymers, metal-polymer composites, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; or any other suitable material. However, this is not meant to be limiting and it is to be understood that the reinforcement layer 37 may include any suitable material, as desired.

The one or more conduits 36 may include any suitable material commonly used in medical devices. In some cases, the conduits 36 may include a material having a higher melt temperature than the outer layer 41 and the inner line 39. Example materials may include, for example, a polymer including but not limited to polyamide (e.g. nylon), polyimide, and polyether ether ketone (PEEK). However, the foregoing materials are merely illustrative and it is contemplated that any suitable materials may be used, as desired.

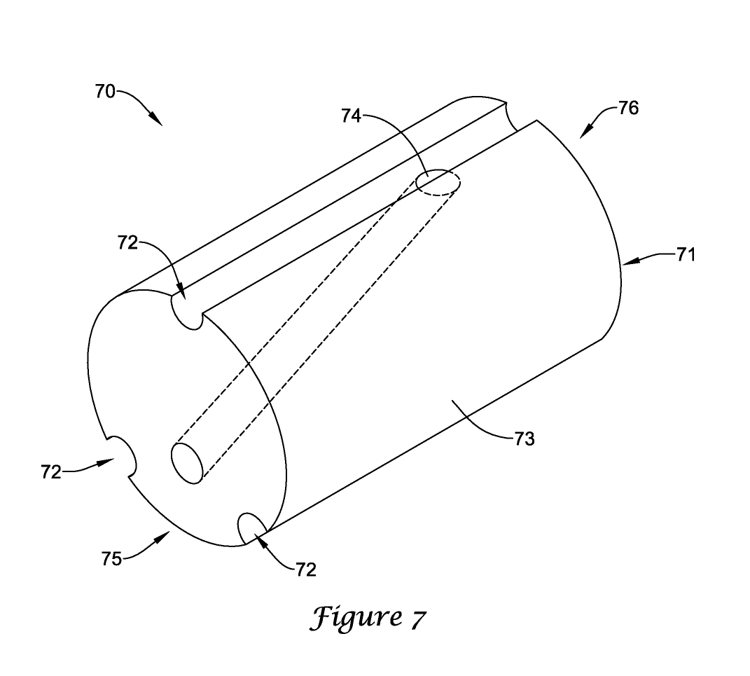

In the illustrative embodiment, one example method of assembling the catheter shaft 20 is as follows. First, the inner line 39 may be assembled over a mandrel (see, for example, mandrel 70 shown in FIG. 7). If conduits 36 are provided, as shown in FIGS. 3 and 4, then, extruded tubes forming conduits 36 may be positioned along the indentations in the polymeric liner 39, which may correspond to indentations in the mandrel 70. If, however, conduits 36 are not desired, the indentations may be filled with a similar material as used in the inner liner 39 or outer layer 41. However, it is contemplated that other materials may be used, as desired. The reinforcement layer 37 can then be positioned over the inner liner 39 and conduits 36. Next, the outer layer 41 can be placed over the reinforcement layer 37.

The layers of the assembled outer tubular member 30 can then be reflowed or bonded together. To do this, in some cases, a compressive heat shrink tube (not shown) can be positioned over the assembled outer tubular member 30. The outer tubular member 30 and heat shrink tube can then heated to a predetermined temperature for a predetermined time that reflows the outer layer 41 and the inner liner 39. The outer tubular member 30 can then be cooled and the heat shrink tube and mandrel can be removed.

In some cases, the inner tubular member 31 can be positioned in the lumen 32 of the outer tubular member 30. The inner tubular member 31 may then be attached to the outer tubular member 30 such as, for example to the distal and/or proximal regions of the outer tubular member 30. When utilized in a balloon catheter, balloon assembly 26 may also be disposed about the distal region 22 of catheter shaft 20. In some cases, a controller, hub, or handle may be coupled to the proximal region 21 of the catheter shaft 20. Further, it is contemplated that other features may be included in the catheter shaft, as desired.

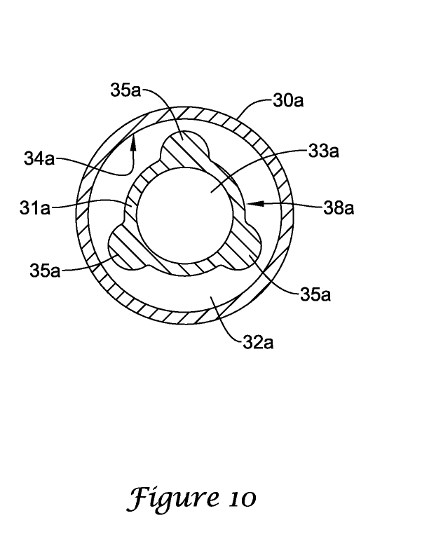

While FIGS. 2, 3 and 4 show the protrusions 35, with or without conduits 36, extending or protruding from the inner surface 34 of the outer tubular member 30, it is contemplated that the protrusions 35 could alternatively or additionally extend from an outer surface 38 of the inner tubular member 31 and into the inflation lumen 32. For example, FIG. 10 shows an inner tubular member 31a including one or more protrusions 35a (without conduits, but it is contemplated that conduits may be provided) disposed in an outer tubular member 30a. Similar to FIG. 2, the catheter shaft of FIG. 10 includes a guidewire lumen 33a defined by inner tubular member 31a and an inflation lumen 32a defined between the inner surface 34a of outer tubular member 30a and outer surface 38a of inner tubular member 31a.

Although not shown in the foregoing embodiments, it is contemplated that balloon catheter 10 may include a supply lumen (not shown) to deliver fluid (e.g. cryogenic fluid) from external source to an interior chamber of the balloon assembly 26. In some cases, a distal end of the supply lumen may include one or more orifices (not shown) configured to release the cryogenic fluid in the interior chamber of the balloon assembly 26. Gas resulting from the cryogenic fluid being released inside the chamber can be exhausted through inflation lumen, such as for example, lumen 32.

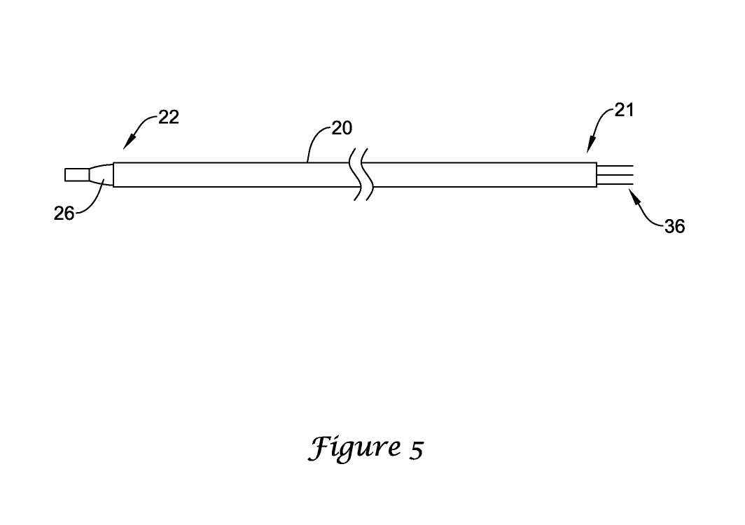

FIG. 5 is an illustrative side view of catheter shaft 20 shown in FIG. 1. As shown, the one or more conduits 36 can be configured to extend proximal from the proximal end of the catheter shaft 20. In some cases, extending the conduits 36 proximally from the catheter shaft 20 may aid in connecting the conduits 36 to, for example, a handle, a controller unit, an electrical board, a pressure transducer, and/or any other external components or equipment, as desired.

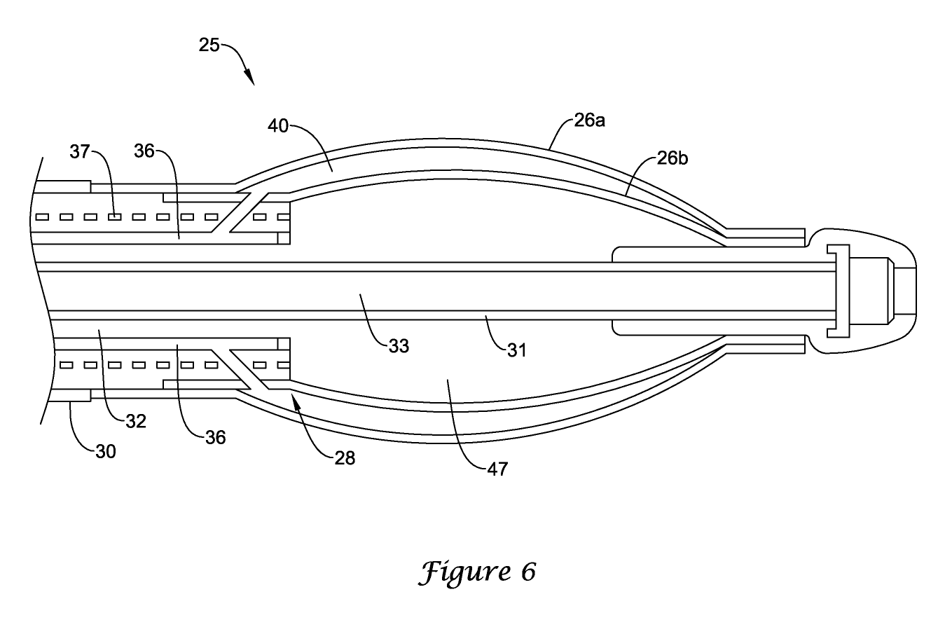

FIG. 6 is a cross-sectional view of an illustrative balloon assembly 26 of the balloon catheter 10 shown in FIG. 1. In the illustrative embodiment, the balloon assembly 26 may include two balloons, an outer balloon 26a and an inner balloon 26b. In the illustrative embodiment, the inner balloon 26a may define a chamber 47 for receiving a fluid (e.g. cryogenic fluid) and the outer balloon 26b may be disposed around the inner balloon 26a. As shown, the chamber 47 of the inner balloon 26b can be in fluid communication with inflation lumen 32. A cooling fluid may be delivered through the inflation lumen 32 in order to inflate the inner balloon 26b and/or outer balloon 26a. As shown, the inner balloon 26b includes a proximal waist that is sealingly secured adjacent to a distal end 28 of the outer tubular member 30 and includes a distal waist that is sealingly secured to the inner tubular member 31 that extends proximally beyond the distal end 28 of the outer tubular member 30. In the illustrated embodiment, cooling fluid may move proximally within the inflation lumen 32 as to allow for removal of cooling fluid and deflation of the inner balloon 26b. However, it is contemplated that other alternative configurations can be provided for supplying and/or exhausting fluid from the balloon chamber 47, such as, for example, providing a separate supply lumen, as discussed previously.

As shown in FIG. 6, a space 40 between the outer balloon 26a and the inner balloon 26b can be in fluid communication with one or more of conduits 36. Although not shown, it is contemplated that only one conduit 36 may be in fluid communication with the space 40 between the outer balloon 26a and the inner balloon 26b, as desired.

In operation, treatment may be effected by positioning the distal end of the balloon catheter 10, and in particular the outer balloon 26a, adjacent a target location in a body. Cryogenic cooling fluid may then be introduced into the chamber 47 of inner balloon 26b. The outer balloon 26a may expand to radially engage the soft tissue and the cooling fluid in the inner balloon 26b can serve to both inflate balloon 26b and to cool the exterior surface of the balloon assembly 26. Example cooling fluids can include, but are not limited to, cryogenic fluids such as liquid nitrous oxide, liquid carbon dioxide, and the like.

In the illustrative embodiment, the dual balloon assembly (e.g. inner balloon 26b and outer balloon 26a) may provide a safety feature of the balloon catheter 10. For example, the outer balloon 26a may function as a safety balloon to prevent the fluid from leaking out of the balloon assembly 26b. That is, in the event that the inner balloon 26b ruptures or otherwise fails, the outer balloon 26a can prevent fluid (e.g., cryogenic fluid) from leaking out of the balloon assembly 26 and contacting body tissue internal to the patient. If cooling fluid does happen to leak out of inner balloon 26b, it could then be removed from the vacuum space 40 via conduit 36. In some embodiments, an automatic fluid shutoff mechanism that monitors containment of the inner balloon 26b can be provided and, if a change is sensed in the vacuum space 40, a shutoff valve to the cooling fluid supply could be closed.

In the illustrative embodiment, balloon assembly 26 may be formed of any suitable material. For example, the balloon assembly 26 may be formed of any suitable non-compliant balloon materials. In other words, the balloon assembly 26 may be constructed to expand to a desired shape when pressurized without elastically deforming substantially beyond the desired shape. Example materials may include, for example, a polymer including but not limited to polyolefin copolymer, polyester, polyethylene teraphthalate, polyethylene, polyether-block-amide, polyamide (e.g. nylon), polyimide, latex, a urethane-family material, neoprene, etc. An example polyether-block-amide is available under the trade name PEBAX®. However, the foregoing materials are merely illustrative and it is contemplated that any suitable materials, either compliant or non-compliant, may be used. In some embodiments, inner balloon 26b and outer balloon 26a may be formed from the same or different material(s), as desired.

FIG. 7 is a perspective view of an illustrative mandrel 70 that may be used in manufacturing the balloon catheter 10 shown in FIG. 1. In the illustrative embodiment, mandrel 70 may include a generally cylindrical body portion 71 extending between a first end 75 and a second end 76. As shown, the body portion 71 may also have a plurality of indentations 72 in the circumferential surface 73, which may correspond to the one or more protrusions 35 of the catheter shaft 20 shown in FIGS. 2-4. In some cases, the plurality of indentations 72 may extend the length of the mandrel 70. However, it is contemplated that the plurality of indentations may extend only a portion of the length of the mandrel according to the design characteristics of the protrusions 35.

As shown in FIG. 7, the mandrel 70 may include on opening 74 extending through the body 71 at an angle thereto. The opening may, for example, extend from end 75 of the body to one of the indentations 72. Opening 74 may enable the conduits 36 to be skived during manufacturing to allow for, for example, temperature and/or pressure sensors to extend out of the conduits 36.

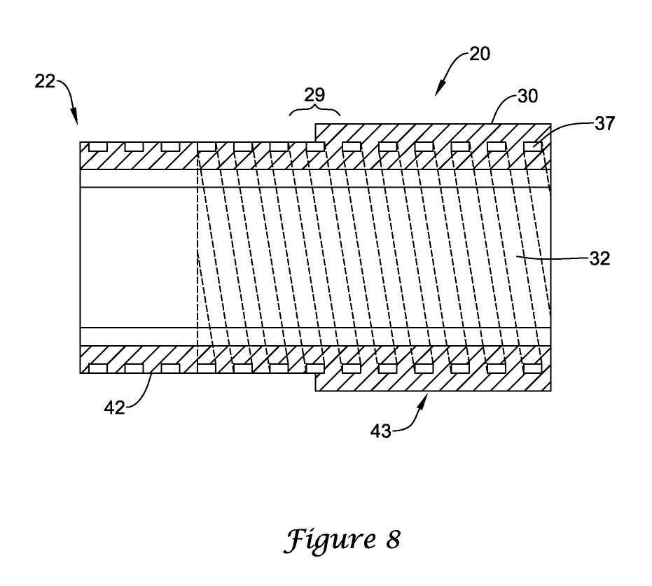

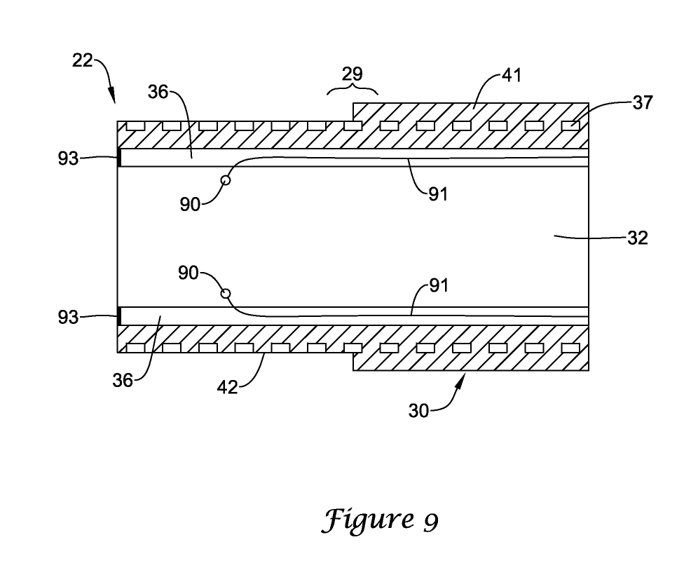

FIGS. 8 and 9 are cross-sectional side views of illustrative distal regions that may be used in the balloon catheter shown in FIG. 1. As shown in FIGS. 8 and 9, the distal region may include a step-down region 29 in the outer diameter of the catheter shaft 20. In some embodiments, the step-down region 29 may be formed by bonding and/or reflowing a tubular member 42 having a relatively small outer diameter with a distal end of another tubular member 43 having a relatively large outer diameter. The illustrative step-down region 29 may provide a reduced outer diameter distal region 22 to the catheter shaft 20 without having to machine or grind down the catheter shaft 20 to accommodate the balloon assembly 26, or any other attachment to the distal region 22.

In one embodiment, an illustrative method of manufacturing balloon catheter 10 having the step-down portion 29 on the distal region 22 of catheter shaft 20 may be similar to the method described above with reference to FIGS. 3 and 4 for manufacturing the catheter shaft 20. However, in addition to the steps provided above, prior to placing the heat shrink tube over the assembled shaft, tube 42, which may have the same inner diameter as outer layer 41 (shown now by reference numeral 43) but a smaller outer diameter, is disposed over the inner liner 39, conduits 36, and reinforcement layer 37. As shown in FIG. 8, a proximal end of the tube 42 can abut a distal end of outer layer 43. The heat shrink tube can then be placed over the assembled outer tubular member 30 and a heat can be applied at a predetermined temperature for a predetermined amount of time. Tube 42 and outer tubular member 43 are thereby reflowed together.

FIG. 9 is similar to the distal region shown in FIG. 8, with the addition of the conduits being skived for placement of various sensors, such as temperature sensors 90. In some embodiment, the conduits 36 may be skived using mandrel 70 shown in FIG. 7. In some embodiments, the conduits 36 may be skived after reflowing the catheter shaft.

As shown in FIG. 9, sensors 90 are configured to extend through the conduits 36 of balloon catheter 10 and exit the conduits 36 and enter the inflation lumen 32 in order to sense temperature or other parameters at various locations along the length of the catheter shaft 20. As shown in FIG. 9, sensors 90 are shown at the same longitudinal location along the catheter shaft 20, but this is not required. It is contemplated that sensors 90 may be positioned at different longitudinal positions, as desired. In some cases, the sensor outputs (e.g. temperature, pressure, etc.) could be entered into a feedback loop that could be used to control the system dynamics of the balloon catheter 10. Although two sensors 90 are shown, it is contemplated that one, three, four, five, six, seven, eight, nine, ten, or any other number of sensors may be provided, as desired.

In one illustrative embodiment, a mandrel, such as mandrel 70, may be positioned in the lumen 32 of the catheter shaft 20. In some cases, this may be performed when assembling the catheter shaft, but this is not required. In this instance, before the mandrel 70 is removed from within a formed outer tubular member 30, a cutting instrument may be inserted into one of the lumens 74 from the first end 75 such that an opening is made or skived in the conduit 36. Either before or after skiving the conduit 36, a sensor 90 and sensor wire 91 can be threaded through the conduits 36 from a proximal end of the conduit 36. The threaded sensor 90 and sensor wire 91 may then be extended, pulled, or otherwise moved through the skived opening and, if the mandrel is still inserted into the lumen 32, down through lumen 74 of the mandrel 70. However, mandrel 70 may be removed prior to extending sensor 90 and senor wire 91 through the skived opening. The sensors 90 and/or sensor wire 91 may then be attached to the outer tubular member 30 as shown in FIG. 9. In some embodiments, a distal end of the conduit 93 may be capped or filled with an adhesive.

Having thus described the preferred embodiments of the present disclosure, those of skill in the art will readily appreciate that yet other embodiments may be made and used within the scope of the claims hereto attached. Numerous advantages of the disclosure covered by this document have been set forth in the foregoing description. It will be understood, however, that this disclosure is, in many respect, only illustrative. Changes may be made in details, particularly in matters of shape, size, and arrangement of parts without exceeding the scope of the disclosure. The invention's scope is, of course, defined in the language in which the appended claims are expressed.

What is claimed is:

1. A catheter comprising:
an outer tubular member including a proximal region, a distal region, and a lumen extending therethrough;
an inner tubular member including a proximal region, a distal region, and a lumen extending therethrough, wherein the inner tubular member is at least partially disposed in the lumen of the outer tubular member;
two or more spacer members positioned between the outer tubular member and the inner tubular member, wherein the two or more spacer members are configured to maintain a gap between the inner tubular member and the outer tubular member to provide a generally uniform temperature distribution for the inner tubular member; and
a balloon assembly including a proximal waist and a distal waist, wherein the proximal waist of the balloon assembly is coupled to the outer tubular member and the distal waist of the balloon assembly is coupled to the inner tubular member.

2. The catheter of claim 1, wherein the two or more spacer members include three or more spacer members.

3. The catheter of claim 1, wherein the two or more spacer members are positioned on an inner surface of the outer tubular member.

4. The catheter of claim 1, wherein the two or more spacer members are positioned on an outer surface of the inner tubular member.

5. The catheter of claim 1, wherein the inner tubular member of the catheter defines a guidewire lumen.

6. The catheter of claim 1, wherein the two or more spacer members are positioned proximal of the balloon assembly.

7. The catheter of claim 1, wherein the two or more spacer members are configured to extend for substantially the entire length of the outer tubular member.

8. The catheter of claim 1, wherein the outer tubular member includes a step-down in outer diameter in the distal region while maintaining a substantially constant inner diameter.

9. The catheter of claim 1, wherein at least one of the two or more spacer members include conduits therethrough.

10. The catheter of claim 9, wherein a sensor and sensor wire extend through at least one conduit.

11. The catheter of claim 10, wherein a portion of the conduit is skived and the sensor and/or sensor wire extend therethrough, wherein the portion is proximal a distal end of the conduit, wherein the distal end of the conduit is capped with an adhesive.

12. A method of manufacturing a catheter body, the method comprising:
assembling a multi-lumen outer tubular member, wherein the multi-lumen tubular member includes:
an inner liner;
a reinforcement layer disposed over the inner liner;
an outer layer disposed over the reinforcement layer; and
two or more conduits disposed between the inner liner and the reinforcement layer, wherein the two or more conduits form two or more radial protrusions on an inner surface of the multi-lumen outer tubular member;
reflowing the inner liner and the outer layer; and
disposing an inner tubular member within the multi-lumen outer tubular member to define a cooling lumen therebetween, wherein the two or more protrusions on the inner surface of the multi-lumen inner tubular member are configured to maintain a gap between the inner tubular member and the multi-lumen outer tubular member.

13. The method of claim 13, wherein the two or more conduits include a higher melting temperature than the inner liner and the outer layer.

14. The method of claim 12, further comprising skiving at least one of the two or more conduits.

15. The method of claim 14, further comprising positioning a mandrel in the outer tubular member, wherein the mandrel has an opening configured to skive the at least one of the two or more conduits.

16. The method of claim 14, further comprising positioning a sensor through the skived conduit.

17. The method of claim 12, wherein the outer layer includes:
a first outer layer member having a first outer diameter;
a second outer layer member having a second outer diameter that is smaller than the first outer diameter;
wherein the first outer layer member and the second outer layer member have substantially the same inner diameter; and
wherein the first outer layer member is positioned proximal of the second outer layer member.

18. The method of claim 17, wherein the first outer layer member and the second outer layer member are bonded together when the inner liner and the outer layer are reflowed.

19. A method of operating a catheter, the method comprising:
providing a balloon catheter including a catheter shaft having proximal region and a distal region, wherein the catheter shaft includes an inner tubular member, an outer tubular member, and a lumen is defined between the inner tubular member and the outer tubular member, wherein the balloon catheter includes a balloon assembly disposed about the distal region of the catheter shaft and in fluid communication with the lumen;
providing a cryogenic fluid through the lumen of the catheter shaft; and
maintaining a substantially uniform temperature distribution throughout a circumference of the inner tubular member when the cryogenic fluid is provided in the lumen of the catheter shaft.

20. The method of claim 19, wherein the substantially uniform temperature distribution is maintained in the inner tubular member by providing three or more protrusions on an inner surface of the outer tubular member or an outer surface of the inner tubular member, wherein the three or more protrusions maintain a gap between the outer surface of the inner tubular member and the inner surface of the outer tubular member.
